(19) United States
(12) Patent Application Publication
(10) Pub. No.: US 2023/0176006 A1
(43) Pub. Date: Jun. 8, 2023

(54) PERFLUORO ACID SENSOR AND METHOD OF USE

(71) Applicants: Manas Ranjan Gartia, Baton Rouge, LA (US); Sushant P. Sahu, Baton Rouge, LA (US); Christopher G. Arges, Baton Rouge, LA (US); Deepra Bhattacharya, Baton Rouge, LA (US); Gokul Venugopalan, Baton Rouge, LA (US)

(72) Inventors: Manas Ranjan Gartia, Baton Rouge, LA (US); Sushant P. Sahu, Baton Rouge, LA (US); Christopher G. Arges, Baton Rouge, LA (US); Deepra Bhattacharya, Baton Rouge, LA (US); Gokul Venugopalan, Baton Rouge, LA (US)

(73) Assignee: Board of Supervisors of Louisiana State University and Agricultural and Mechanical College, Baton Rouge, LA (US)

(21) Appl. No.: 18/060,201

(22) Filed: Nov. 30, 2022

Related U.S. Application Data
(60) Provisional application No. 63/285,301, filed on Dec. 2, 2021.

(51) Int. Cl. G01N 27/407; G01N 27/404; G01N 27/30

(52) U.S. Cl. CPC G01N 27/4074; G01N 27/301; G01N 27/4045

(57) ABSTRACT

Disclosed herein is a sensor for measuring perfluoroalkyl acids and/or polyfluoroalkyl acids. The sensor includes a working electrode, a counter electrode and optionally a reference electrode. The working electrode includes a film disposed on the surface of the working electrode and the film includes a perfluorinated anion exchange ionomer. A method of using the sensor to detect perfluoroalkyl acids and/or polyfluoroalkyl acids is also described.

### CROSS-REFERENCE TO RELATED APPLICATIONS

The present application claims priority to U.S. Provisional Application No. 63/285,310, filed Dec. 2, 2021, which is incorporated by reference herein in its entirety.

### STATEMENT REGARDING FEDERALLY SPONSORED RESEARCH OR DEVELOPMENT

This invention was made with government support under Award No. 1703307 awarded by the National Science Foundation and Award No. DE-SC0018989 awarded by the United States Department of Energy. The government has certain rights in the invention.

### BACKGROUND OF THE INVENTION

Perfluoroalkyl substances (PFASs) pose a significant health threat to humans at trace levels. Because of ubiquity of perfluoroalkyl substances across the globe, there has been intense efforts to rapidly quantify perfluoroalkyl substances in the environment while also limiting their release.

Perfluoroalkyl and polyfluoroalkyl substances (PFAS) are a by-product of many materials that make up our everyday life (e.g., fire-fighting foams, lubricants, non-stick cookware, coatings for textiles, carpet, paper, food packaging etc.). These chemicals, and their high molecular weight polymer analogues (e.g., polytetrafluoroethylene), are termed “Forever Chemicals” because of their exceptional chemical stability in the presence of oxidizers and extreme pH values. The miracles wrought by perfluorinated polymers have unfortunately led to the release of PFAS into the environment. As such, PFAS poses serious remediation challenges and threats to public health. (Hopkins et al., Journal American Water Works Association 2018, 110 (7) 13-28; Kwiatkowski et al., Environmental Science & Technology Letters 2020, 7 (8), 532-543) For example, bioaccumulation of PFAS in humans are well-documented and include cancer (Barry et al., Environmental Health Perspectives 2013, 121 (11-12), 1313-1318), estrogen disruption (Xin et al., Environmental Science & Technology 2019, 53 (14), 8371-8380), protein misfolding (Sheng et al., Archives of Toxicology 2018, 92 (1), 359-369), birth defects and other health risks (Grandjean et al., JAMA 2012, 307 (4), 391-397, Braun et al., Obesity 2016, 24 (1), 231-237, Saikat et al., Environmental Science: Processes & Impacts 2013, 15 (2), 329-335, Gomis et al., Environment International 2018, 113, 1-9, Sunderland et al., Journal of Exposure Science & Environmental Epidemiology 2019, 29 (2), 131-147). These challenges have spurred research in technologies for PFAS sensing (Rodriguez et al., Micromachines 2020, 11 (7), 667), removal (Ateia et al., Environmental Science & Technology Letters 2019, 6 (12), 688-695; Kumarasamy et al., ACS Central Science 2020, 6 (4), 487-492; Ji et al., Journal of the American Chemical Society 2018, 140 (40), 12677-12681) and destruction upon sequestration (Saleh et al., Environmental Science: Water Research & Technology 2019, 5 (2), 198-20; Cui et al., Environmental Science & Technology 2020, 54 (7), 3752-3766; Chaplin et al., Environmental Science: Processes & Impacts 2014, 16 (6), 1182-1203).

Traditional detection methods for PFAS in environmental matrices rely upon on complex machinery such as LC-MS and HPLC. These instruments are bulky and expensive and require time consuming sample preparation. Although accurate and reliable, they cannot quantify PFAS contaminants within in a minute or less. Because field workers may wish to identify PFAS concentrations rapidly or to sense a containment vessel leak, there is a need to develop economical and straightforward PFAS sensing technologies with broad applicability.

Electrochemical detection platforms (Alam et al., ACS sensors 2020, 5 (2), 412-422) are an enticing prospect for PFAS sensing in the field as they are portable, robust, and economical. Electrochemical sensors have been shown to detect PFAS in a diverse class of matrices ranging from environmental water to blood serum with concentration of perfluorooctanoic acid (PFOA) ranging from 15.7 to 128 ppb, especially in a community residing close to PFAS production facility (Karstadt et al., Environmental Health Perspectives 2007, 115 (10), A486-A487). A notable electrochemical sensor by Karimian et.al (ACS sensors 2018, 3 (7), 1291-1298) showed a level of detection (LOD) of 0.04 nanomolar (nM) (17 ppt) for PFOA in deionized water using template-assisted molecularly imprinted polymer. This sensor provides indirect detection of PFOA because it relies upon ferrocenecarboxylic acid redox active probe. The electrochemical signal from this probe is quenched by the presence of PFOA. The strong affinity of PFOA for common electrode materials, like gold, necessitate molecularly imprinted polymer to stymie PFOA adsorption. Without a porous polymer coating, any amount of PFOA would quickly saturate the electrode response. There are also other ferrocene-based redox probes for electrochemical sensing of PFOA - some with molecularly imprinted polymers (Glasscott et al., Environmental Science & Technology Letters 2020, 7 (7), 489-495; Clark et al., ACS sensors 2020, 5 (11), 3591-3598). In one of these other demonstrations (Glascott et al.), GenX which is also a similar class of perfluoroalkyl acids was quantified in actual river water at concentrations approaching ~ 1 picomolar (pM). A drawback of these indirect electrochemical sensors for PFAS is that they require the addition of redox active probes. The need to carry chemical reagents with the sensor is inconvenient for portable electronics, like smart phones, and can complicate the sensor design. A new approach is needed.

### SUMMARY OF THE INVENTION

In one aspect, the present invention relates to a sensor for measuring perfluoroalkyl acids and/or polyfluoroalkyl acids comprising: a working electrode; and a counter electrode; wherein the working electrode comprises a film disposed on the surface of the working electrode; and wherein the film comprises a perfluorinated anion exchange ionomer. In one embodiment, the sensor further comprises a reference electrode. In one embodiment, the film has a thickness of 5 to 500 nm. In one embodiment, the working electrode comprises cobalt, platinum, palladium, gold, iron, aluminum, copper, nickel, zinc, silver, or an alloy comprising one or more of the foregoing. In one embodiment, the working electrode, counter electrode and optional reference electrode are screen printed on a ceramic, glass, quartz, polymer, or hydrogel substrate. In one embodiment, the working electrode further comprises a nanoscale electrocatalyst. In one embodiment, the working electrode comprises nanoparticles. In one embodiment, the working electrode and counter electrode are interdigitated.

In one embodiment, the perfluorinated anion exchange ionomer comprises a polymer comprising perfluoro groups and at least one cationic group tethered to the polymer comprising the perfluoro groups. In one embodiment, the perfluoro groups are located on a backbone of the polymer, on a side chain or both. In one embodiment, the at least one cationic group is an ammonium, a phosphonium, a sulfonium, an organometallic cation, or a combination thereof. In one embodiment, the cationic group includes one or more of the following structures:

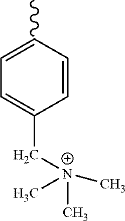

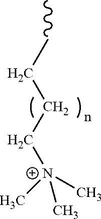

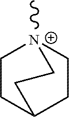

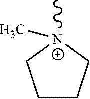

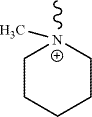

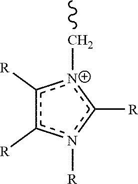

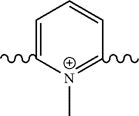

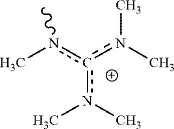

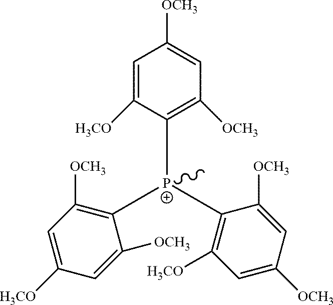

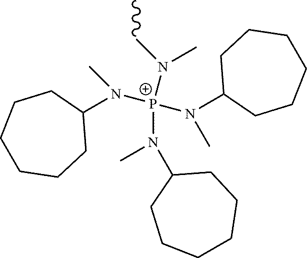

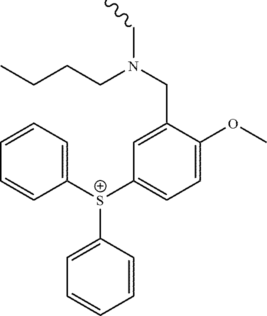

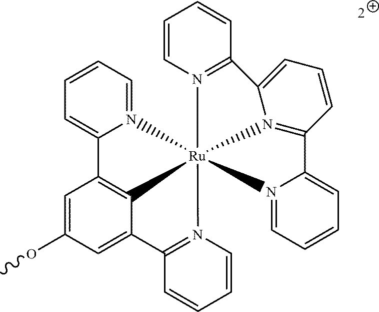

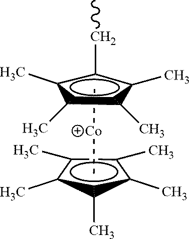

wherein the wavy line indicates the attachment point to the polymer;
each occurrence of R is independently selected from the group consisting of hydrogen, deuterium, fluorine, C₆-C₁₈ aryl, and C₁-C₁₀ alkyl; and
n is an integer having a value between 1 and 100.

The invention also relates to a containment vessel comprising the sensor for measuring perfluoroalkyl acids and/or polyfluoroalkyl acids, including a containment vessel comprising a series of point sensors functionally connected to an alarm system.

In another aspect, the present invention relates to a method of detecting perfluoroalkyl acids and/or polyfluoroalkyl acids comprising the steps of providing a sample for measurement; contacting the sample with a sensor; and determining the presence of perfluoroalkyl acids based on the response of the sensor to the sample. In one embodiment, the method further comprises the step of acidifying the sample to a pH of 1 to 3. In one embodiment, the sensor is a sensor for measuring perfluoroalkyl acids and/or polyfluoroalkyl acids described herein.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 shows an electrode having a nanocatalyst.

FIG. 2 shows an interdigitated electrode having a nanocatalyst.

FIG. 3A illustrates preparation of perfluorinated anion exchange ionomer (PFAEI) coating on a commercial screen printed electrode (SPE) configuration;

FIG. 3B illustrates a proposed mechanism for PFAA sensing with PFAEI coating on the SPE; and

FIG. 3C illustrates preparation of perfluorinated anion exchange ionomer (PFAEI) coating on a different commercial screen printed electrode (SPE) configuration.

FIG. 4A shows square wave voltammetry (SWV) of platinum wire working electrode data with different ionomers at PFOA concentration of 7.68 µM;

FIG. 4B shows normalized current response with different ionomers at -0.40 V vs Ag/AgCl;

FIG. 4C shows square wave voltammetry (SWV) of wire data with PFAEI coating with various PFOA concentrations at -0.40 V vs Ag/AgCl;

FIG. 4D shows normalized current responses as a function of PFOA for a given cathodic peak potential for PFAEI ionomer coated Pt and neat Pt electrode @ -0.40 V vs Ag/AgCl; and

FIG. 4E shows bar graphs with different interfering anions for PFAEI coated platinum electrode measured at applied potential of -0.47 V vs Ag/AgCl using chronoamperometry.

FIG. 5A shows square wave voltammetry (SWV) data of SPE with PFAEI coating interfaced with buffered deionized water;

FIG. 5B shows SWV data of SPE with PFAEI coating interfaced with drinking water;

FIG. 5C shows normalized current response from chronoamperometry experiments at potential values that gave peak current for buffered DI water; and

FIG. 5D shows normalized current response from chronoamperometry experiments at potential values that gave peak current for drinking water.

FIG. 6 shows normalized cathodic current response measured at -0.4 Volt as a function of PFOA concentration using 3 wt% PFAEI coated Pt wire electrodes investigated at different pH values of the supporting electrolyte adjusted using dilute NaOH (0.75 M) or HClO₄.

FIGS. 7A and 7B shows cyclic voltammetry (7A), and corresponding normalized current response (7B) measured at -0.68 V Vs Ag as a function of PFOA concentration using PFAEI coated Pt Dropsens electrode (DS-550) in deionized water.

FIGS. 7C and 7D show square wave voltammetry (SWV) results (7C) and chronoamperometry response (7D) seen at applied potential step of -0.68 V Vs Ag in the presence of 15 nM PFOA using PFAEI coated Pt Dropsens electrode (DS-550) in deionized water.

FIGS. 8A and 8B show cyclic voltammetry (8A), and corresponding normalized current response (8B) measured at -0.85 V Vs Ag as a function of PFOA concentration using PFAEI coated Pt Dropsens electrode (DS-550) in drinking water; and

FIG. 8C shows SWV response in simulated drinking water sample spiked with 15 nM PFOA employing PFAEI coated Pt Dropsens (DS-550) electrodes.

FIGS. 9A and 9B show a comparison of PFOA sensor performance showing limit of detection (LOD) and dynamic range of PFOA detection through normalized cathodic current response obtained from voltammetry measurements as a function of PFOA concentration using PFAEI coated Pt screen-printed electrode configurations from Dropsens (DS-550) and Pine electrodes in deionized water (9A) and in drinking water samples (9B).

FIG. 10 shows normalized cathodic current responses measured at -0.40 V employing PFAEI coated Pt wire electrodes by using different concentrations of PFAEI ionomer loading solutions in the detection of PFOA at different PFOA concentrations.

FIG. 11 shows experimentally measured chronoamperometry curves measured at applied potential step of -0.55 V (Left) using PFAEI coated Pt wire electrode as PFOA sensor at different PFOA analyte concentrations in deionized water.

FIG. 12 shows cyclic voltammograms showing increasing current response as function of PFOA concentration in deionized water using PFAEI coated Pt wire electrode sensor.

FIG. 13 shows chronoamperometry response measured at applied potential step of -0.8 V Vs Ag/AgCl due to increasing PFOA concentrations using PFAEI coated Pt wire electrodes in drinking water.

FIGS. 14A-C show chronoamperometry responses measured at applied potential step of -0.85 V Vs Ag/AgCl using PFAEI coated Co wire electrodes in presence of interfering anions with concentrations up to 50 µM of nitrate (NO₃⁻) (14A), orthophosphate (H₂PO₄⁻) (14B), and sulfate (SO₄²⁻) ion with and without addition of 40 µM PFOA (14C).

FIGS. 15A-C show Chronoamperometry response measured at applied potential step of -0.47 V Vs Ag/AgCl employing PFAEI coated Pt wire electrodes in presence of 50 mM orthophosphate (H₂PO⁻₄) (15A), nitrate (NO₃⁻) (15B) and sulfate (SO₄²⁻) (15C) as interfering anions with and without addition of 40 µM PFOA.

FIGS. 16A and Bshow square wave voltammetry of wire data with PFAEI coating with various PFOS concentrations vs Ag/AgCl (16A) and normalized current responses as a function of varying PFOA and PFOS concentrations measured at a given cathodic peak potential for PFAEI ionomer coated Pt wire electrodes (16B).

FIGS. 17A and Bshow a comparison of PFAEI coated metal electrode (Co and Pt) sensitivity in PFOA detection using metal wire electrodes. FIG. 17A shows current density after normalization with respect to geometric surface area of wire electrodes. FIG. 17B shows as normalized current response at specified potential observed in PFOA detection as a function of increasing PFOA concentration.

### DETAILED DESCRIPTION

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Perfluoroalkyl acids are organic compounds in which all C—H bonds are replace with C—F bonds. Polyfluoroalkyl acids contain C—F bonds and at least one C—H bond. As used herein the term PFAA refers to perfluoroalkyl acids, polyfluoroalkyl acids, or a combination thereof.

A rapid and direct electrochemical sensor for PFAA is obtained through the appropriate selection of a selective ionomer coating (i.e., a perfluorinated anion exchange ionomer (PFAEI)) and an electrode such as a wire electrode or screen printed electrode (SPEs). The electrode may be an interdigitated electrode. The ability to perform direct sensing of PFAA is attributed to both electrostatic and van der Waal interactions between the PFAEI coating and PFAA. These interactions have been inferred by testing comparative ionomer coatings such as a hydrocarbon anion exchange ionomer (quaternary benzyl pyridinium chloride poly(arylene ether sulfone) (QPPSf)), hydrocarbon cation exchange ionomer (sulfonic acid polyether ether ketone) (SPEEK)), and a perfluorinated cation exchange ionomer (Nafion™ - a type of perfluorosulfonic acid). By testing the sensor with different ionomer electrode coatings, it was inferred that PFAEI favors PFAA anions over other competing anions in solution through a combination of electrostatic and van der Waal interactions. The comparative ionomer coatings were ineffective at sensing PFAA and highlight the need for anion-exchange ionomers comprising perfluoro groups in the backbone, side chains or both to promote van der Waal interactions and a cationic group for PFAA anion exchange. The PFAEI coating is described in greater detail below.

Described herein is an electrochemical sensor with a selective perfluorinated anion exchange ionomer (PFAEI) coating for direct sensing of PFAA. Notably, the sensor operates without the need for redox probes and has a limit of detection (LOD) less than or equal to 7 ppb, or, less than or equal to 6 ppb, or, less than or equal to 5 ppb in buffered deionized water as well as drinking water. Sensitivity for polyfluoroalkyl acids may be related to the degree of fluorination. Exemplary detectable PFAA materials include perfluorooctanoic acid (PFOA), perfluorooctane sulfonic acid (PFOS), GenX (CF₃CF₂CF₂OCF(COOH)CF₃), and combinations thereof. The sensor is specific for the detection of PFAA materials in the presence of potentially interfering anions such as chloride, nitrate, sulfate, and phosphate.

The sensor comprises a working electrode, a counter electrode and optionally a reference electrode. The working electrode may comprise a metal or metal alloy such as cobalt, platinum, palladium, gold, iron, aluminum, copper, nickel, zinc, silver, or an alloy comprising one or more of the foregoing. The metal or metal alloy may be in the form of nanoparticles. The nanoparticles may have the shape of a sphere, rod, star, cube, oval, cage, or have a core-shell structure. When the nanoparticles have a core-shell shape, they may include a core comprised of the metal or metal alloy and a thin (nanoscale) protective shell that may be comprised of one or more electrically conductive oxide or nitride materials such as SiO₂, TiO₂, Al₂O₃, SiN, AlN, or TiN. The shell may be porous. The nanoparticles may be disposed on and/or embedded in two-dimensional electrically conductive materials (graphene, MoS₂, BN, WSe₂ or black phosphorous) or carbon cloth or mesh made of platinum, gold, aluminum, cobalt, copper, nickel foam, carbon fiber or a combination thereof.

The working, electrode, counter electrode and optional reference electrode may be a wire electrode or screen printed on a ceramic, glass, quartz, polymer (such as PET, PTFE, polystyrene, silk) or hydrogel substrate as is known in the art. Exemplary electrodes are available from Pine Research and Metrohm Dropsens.

A film is located on the working electrode. The film comprises a perfluorinated anion exchange ionomer. Perfluorinated anion exchange ionomers are described in the following references: Park et al., ECS Trans. 2017, 80, 957, Kim et al.,Macromolecules 2013, 46, 7826, Lee et.al., Membranes 2020,10 , 306, Divekar et.al, J. Polym. Sci., Part B: Polym. Phys. 2019, 57, 700, Ugo et.al Anal. Chim. Acta 1993, 273, 229. The film may have a thickness of 5 to 500 nanometers (nm), or, 5 to 400 nm, or 10 to 300 nm. The perfluorinated anion exchange ionomer comprises a polymer comprising perfluoro groups and at least one cationic group tethered to the polymer comprising the perfluoro groups. The perfluoro groups may be located on the backbone of the polymer, on a side chain or both. The at least one cationic group may be an ammonium, a protonated amine, a phosphonium, a sulfonium, an organometallic cation, or a combination thereof. Exemplary cationic groups include one or more of the below structures. The wavy line indicates the attachment point to the polymer and n is an integer having a value between 1 and 100. In one embodiment, n has a value between 1 and 10. In one embodiment, n has a value between 3 and 6. In one embodiment, n has a value of 1, 2, 3, 4,5, 6,7, 8, 9, or 10. Each occurrence of R is independently selected from the group consisting of hydrogen, deuterium, fluorine, C₆-C₁₈ aryl, and C₁-C₁₀ alkyl. In some embodiments, each R represents hydrogen or methyl.

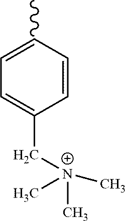

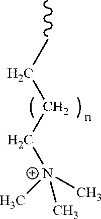

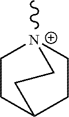

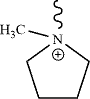

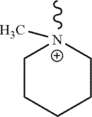

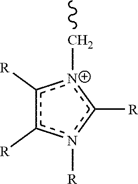

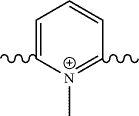

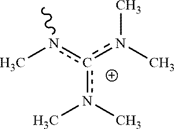

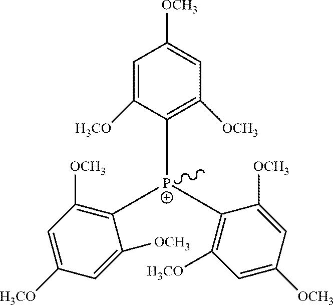

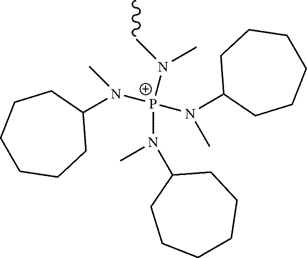

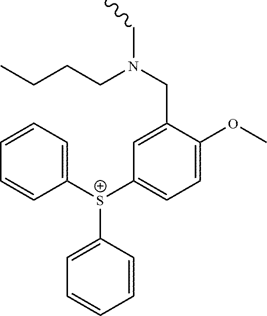

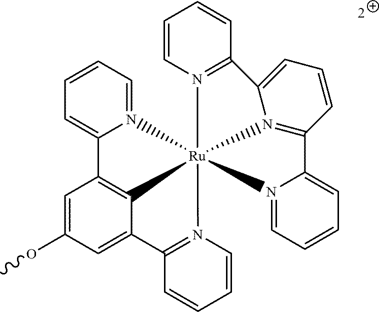

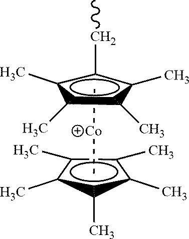

The cationic group may include fluorinated moieties. For example, a CH₃ may be replaced with a CF₃ and/or a CH₂ may be replaced with a CF₂. The choice of the cationic group or combination of cationic groups may be chosen relative to the PFAA material or materials being detected. Without being bound by theory it is believed that there may be a relationship between cationic group on the perfluorinated anion exchange ionomer and the pKa of the PFAA material.

The perfluorinated anion exchange ionomer may consist essentially of perfluoro groups and cationic groups. In this context, “consists essentially of” allows for the presence of linking groups between the perfluoro portion of the polymer and the cationic group(s) but the ionomer otherwise consists of perfluoro groups and cationic groups. The ionomer may have ionexchange capacity of 0.9 milliequivalents/gram of ionomer to 2.5 milliequivalents/gram of ionomer.

It is also contemplated that the perfluorinated anion exchange ionomer may also comprise CH₂ groups in the backbone of the polymer.

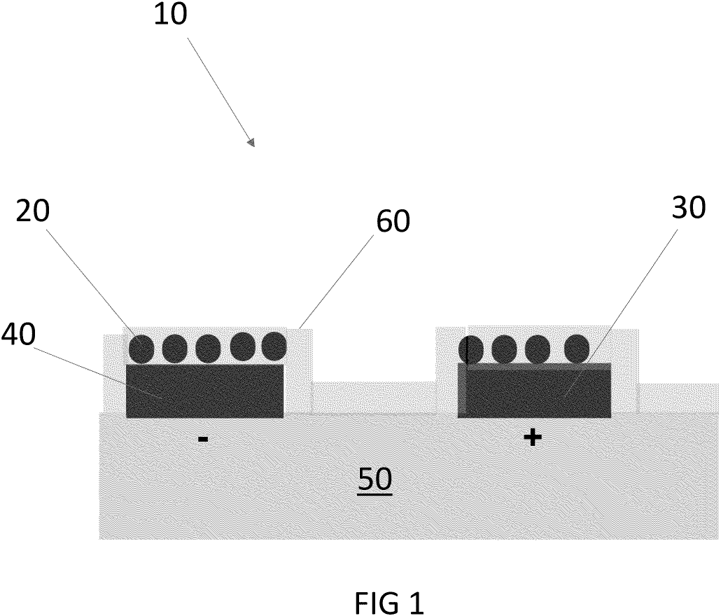

The working electrode may further comprise a nanoscale electrocatalyst such as platinum located between the surface of the working electrode and the perfluorinated anion exchange ionomer coating. Exemplary nanoscale electrocatalysts include platinum, palladium, iridium, gold, copper, iron, cobalt, nickel, zinc, tin, and oxides thereof. The nanoscale electrocatalyst may be compositionally the same as or different from the working electrode. FIG. 1 shows an embodiment of a portion of a working electrode 10 utilizing a nanoscale electrocatalyst. Working electrode 10 has a nanoscale electrocatalyst 20 located on a cathode 30 and an anode 40. The cathode 30 and anode 40 are located on a substrate 50. A PFAEI coating 60 is disposed on the nanoscale electrocatalyst 20, the cathode 30 and the anode 40. Nanoscale electrocatalyst and their deposition are described in Bhattacharya et al., Small 2021, 2100437.

It is further contemplated that the working electrode and counter electrode may be interdigitated and may optionally comprise a nanoscale electrocatalyst for increased surface area. Nanoscale, as used herein, refers to structures having dimensions less than 100 nm in lateral feature sizes. The dimension of the interdigitated electrodes may be 0.5 micrometers (µm) to 5 millimeters (mm) in teeth width, 0.1 mm to 3 centimeters (cm) in teeth length, and the teeth can be embedded and placed topographically on the plastic or substrate surface as described by Arges et al., J. Mater. Chem. A, 2017 and Sharon et al., J. Electrochem. Soc. 2018.

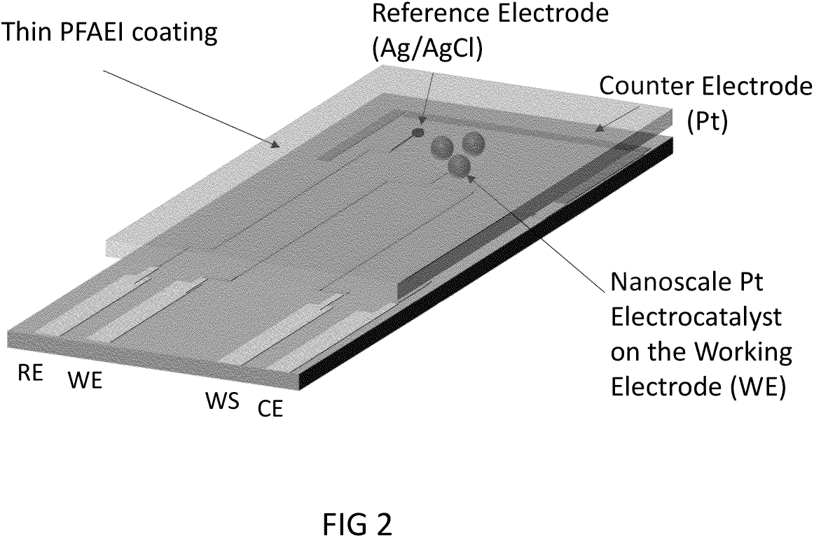

FIG. 2 is a representation of interdigitated electrodes.

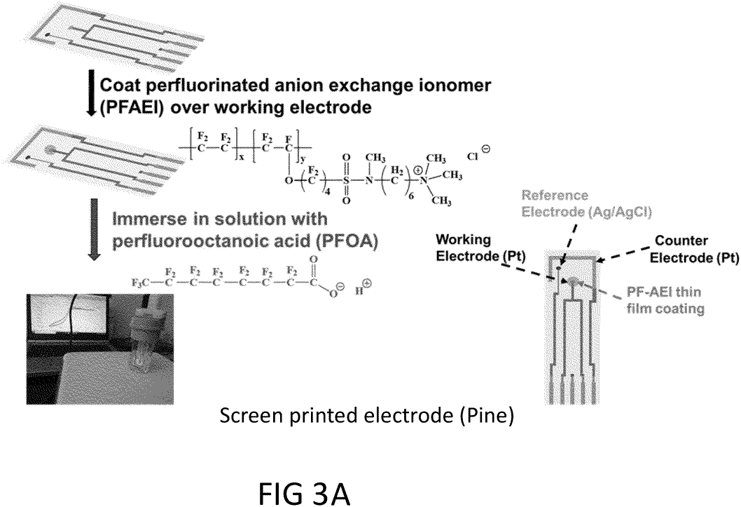

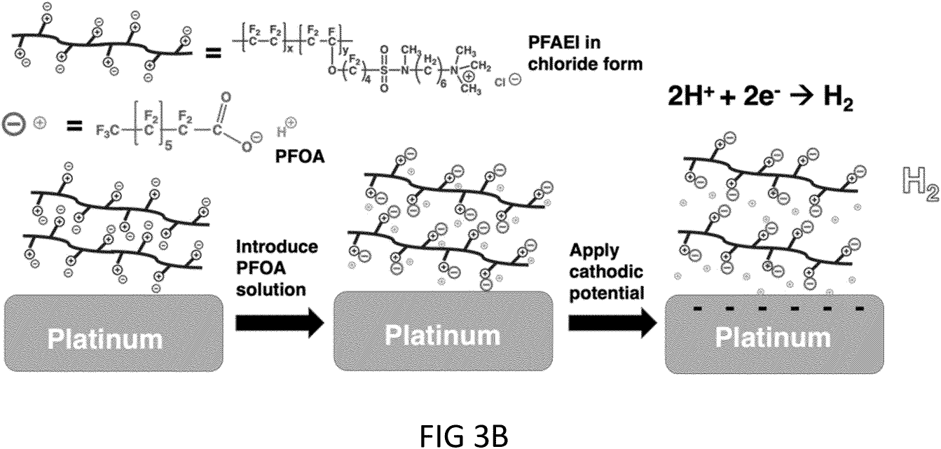

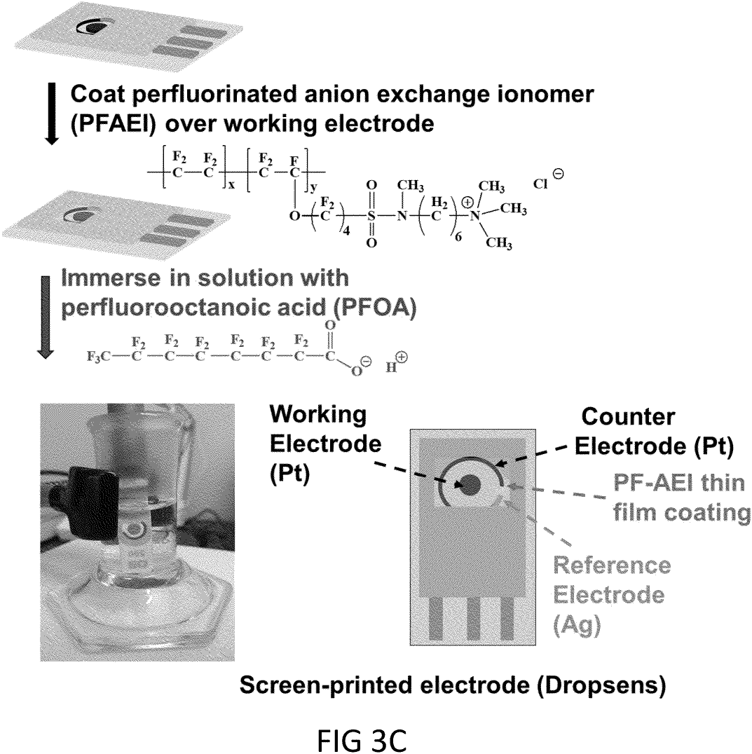

FIG. 3A illustrates a screen-printed electrode (SPE) with a PFAEI coating for PFAA sensing. Because of the sensor’s planar nature, they are conducive for deposition of PFAEI and other ionomer coatings on the electrodes. One advantage of the PFAEI coating is that it prevents direct PFAA adsorption on the electrode surface. With respect to the sensing mechanism, it is postulated that the tethered quaternary cationic moiety in PFAEI exchanges with PFAA anion in solution. The perfluoro-nature of the PFAEI backbone is conducive for PFAA anion adsorption and ion-pairing with the tethered quaternary ammonium. Hence, both van der Waal and electrostatic interactions work cooperatively for promoting PFAA uptake in the PFAEI. The proton released from the exchanged PFAA can permeate the PFAEI as the quaternary ammonium groups are screened by the PFAA anion (i.e., poorly dissociated) and protons break and form bonds with water. Hence, the protons permeate through anion exchange materials despite other cations resisting permeation due to Donnan exclusion. The protons then migrate to the negatively biased electrode across the PFAEI-PFAA anion material to undergo the hydrogen evolution reaction (HER). The current response from HER is used to detect and quantify PFAA in solution. FIG. 3B illustrates the PFAA anion exchange with PFAEI, proton permeation in the PFAEI to the cathode, and the HER process. FIG. 3C illustrates a screen-printed electrode (SPE) with a PFAEI coating for PFAA sensing and having a slightly different configuration than the screen printed electrode shown in FIG. 3A.

Also disclosed is a method of detecting PFAA comprising providing a sample for measurement, optionally acidifying the sample to a pH of 1 to 3, contacting the sample with the sensor, and determining the presence of PFAA based on the response of the sensor to the sample.

The sensor and method can be used to detect leaks in containment vessels. For example, a PFAEI coated cobalt mesh can be located on the exterior of a containment vessel to serve as a working electrode and functionally connected to a counter electrode located in or on the interior of the containment vessel. In another embodiment a series of point sensors can be located on the exterior of a containment vessel and functionally connected to an alarm system. The sensor and method can also be used to evaluate the permeance of PFAS across barrier materials.

### EXPERIMENTAL SECTION

### Materials

Platinum electrode wire (0.5 mm OD, 99.99%) and screen-printed platinum electrodes were obtained from Pine Research Instrumentation, Inc. Perfluorooctanoic acid (PFOA, 96%) and perchloric acid (ACS grade 70%) were purchased from Sigma Aldrich. Nafion™ dispersion (20 wt% in alcohol-water mixture) was obtained from Ion Power. The perfluorinated anion exchange ionomer (PFAEI) was supplied from the National Renewable Energy Laboratory (NREL). This material was prepared from the sulfonyl fluoride form of perfluorosulfonic acid membrane precursors made by 3 M (Park et al., ECS Trans. 2017, 80, 957- 966). Sulfonated polyether ether ketone (SPEEK, degree of functionalization value, DF = 0.6) and Udel® poly(arylene ether sulfone) featuring quaternary benzyl pyridinium chloride (QPPSf) were synthesized in house as described in Venugopalan et al., ACS Applied Energy Materials 2020, 3, 573-585 and Kole et al., J. Mater. Chem. A 2021, 9, 2223-2238. Methanol (MeOH, > 99.8 %), n-methyl-2-pyrrolidone (NMP) (> 99.0 %), isopropanol (≥ 99.5 %), hydrochloric acid (HCl, ACS grade, 37%) were obtained from VWR and N,N-dimethylacetamide (DMAc) (ACS grade) from Alfa Aesar. Potassium sulphate (K₂SO₄, ACS grade) and potassium nitrate (KNO₃, ACS grade) was acquired from VWR. Potassium dihydrogen phosphate, monobasic (KH₂PO₄, ACS grade) was purchased from Ward Science. Sodium hydroxide (NaOH, ACS grade) was obtained from Fisher Chemicals. Deionized water produced from Millipore Milli-Q with 18.0 MΩ resistivity and was utilized for preparing the solutions. Local tap drinking water (LA, Baton Rouge) were used as drinking water source. All chemicals were of analytical grade and used as received.

The chemical structures of the various ionomer coatings used in the Examples is shown below.

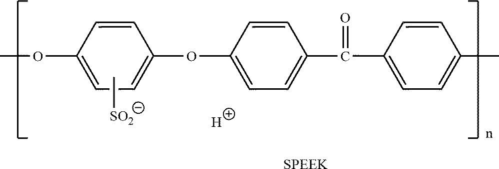

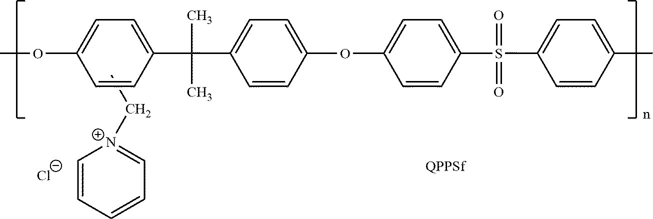

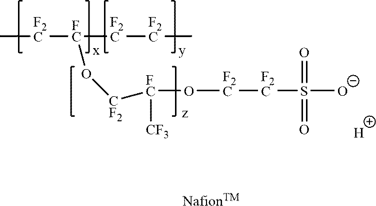

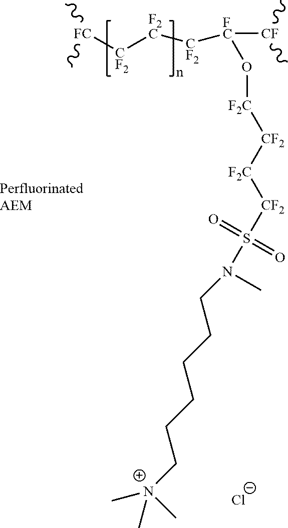

### Preparation of Ionomer Coated SPEs for PFOA Detection

Ceramic SPEs with platinum working (3.14 mm²) and counter electrodes and a silver-silver chloride reference electrode (Ag/AgCl) were acquired from Pine Research. The ionomer (3 wt% in a mixture of isopropanol (IPA) and N,N-dimethylacetamide (DMAC)) was drop casted on the SPE followed by drying at room temperature and removing solvent further by vacuum drying for 5 minutes at room temperature. Thickness of the ionomer coated on the substrate was determined via ellipsometry and was about 50 to 70 nm.

### Preparation of PF-AEI Coated Wire Electrodes for PFOA Detection

Pt metal wire electrode (OD = 0.5 mm; approximate total surface area = 0.58 cm²) were submerged in solution of 3 wt% ionomer in a mixture of isopropanol (IPA) and N,N-dimethylacetamide (DMAc) for three minutes followed by vacuum drying for five minutes to obtain PFAEI coated metal wire electrodes.

### Electrochemical Sensing Experiments With Wire Electrodes

A Potentiostat/Galvanostat, Gamry Instruments 1010E, was employed for square wave voltammetry (SWV) and chronoamperometry experiments. PFOA sensing was demonstrated using a three-electrode configuration, with a platinum wire serving as a working electrode, platinum counter electrode, and a Ag/AgCl reference electrode. All experiments used supporting electrolyte buffer containing 70 mM HClO₄ and 33 mM NaOH with PFOA in a concentration range of 0.76 µM to 100 µM at pH ~ 1.5. The normalized current response i/i* is used to represent the sensor response versus PFOA concentration (note: i* is the current response with no PFOA present in the solution). The chronoamperometry measurements were performed in the same concentration range by applying a cathodic step potential of -0.47 V vs Ag/AgCl for the platinum wire coated with PFAEI for a period of 60 seconds.

### Electrochemical Sensing Experiments With SPEs

SWV were carried out on the Pine SPEs immersed into the solution that contained PFOA of varying concentration in the range from 0 to 500 nM. A supporting electrolyte matrix before the addition of PFOA contained perchloric acid (HClO₄) at 70 mM and sodium hydroxide at 33 mM. This resulted in a solution pH of 1.5. All SWV experiments are conducted in the potential window from 0 to -1.0 V vs Ag/AgCl reference electrode using a 50 mV amplitude, potential step 0.75 mV and frequency of 25 Hz. Mild stirring was applied during SWV experiments. Chronoamperometry measurements were performed by applying a cathodic step potential of -0.76 V on SPEs for a period of 60 seconds. The cathodic peak potential in different experiments is determined from voltammetry measurements.

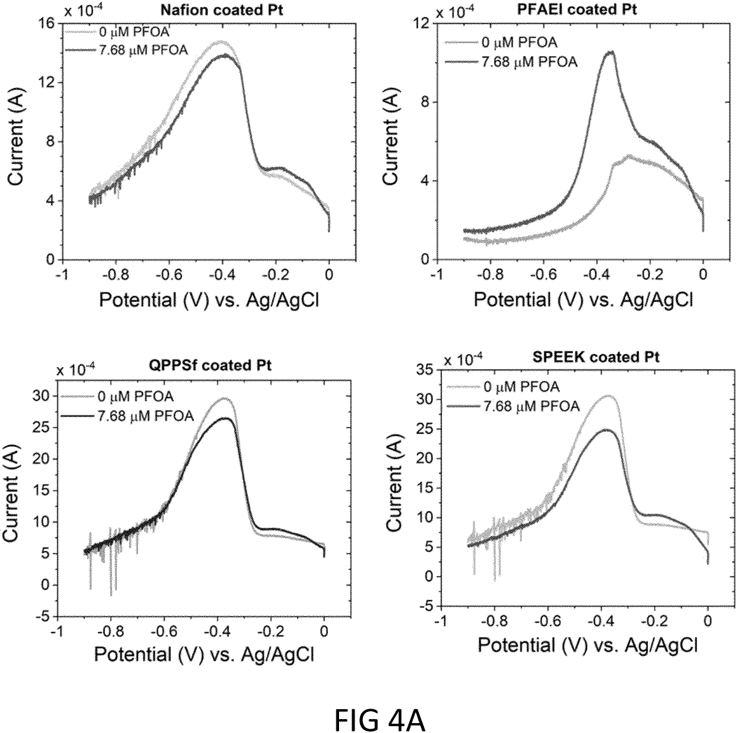

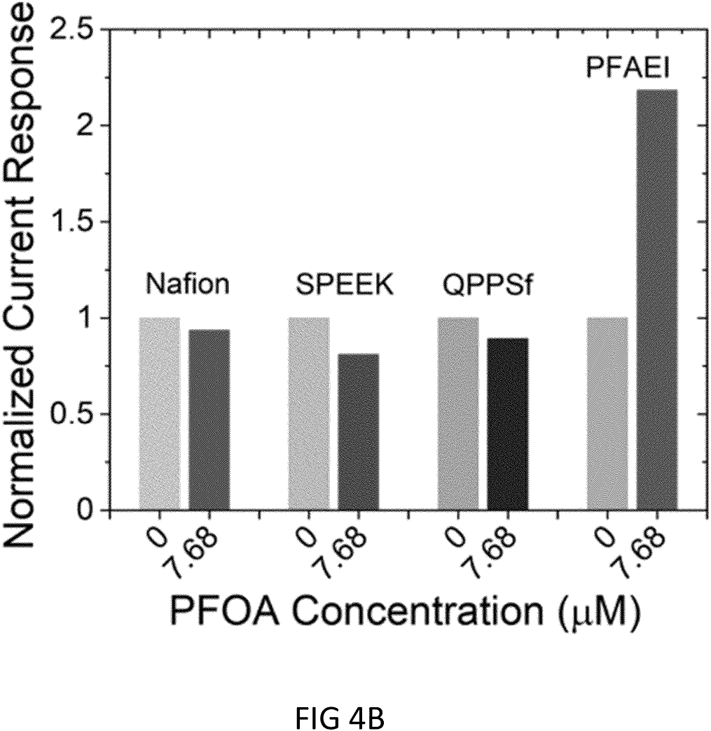

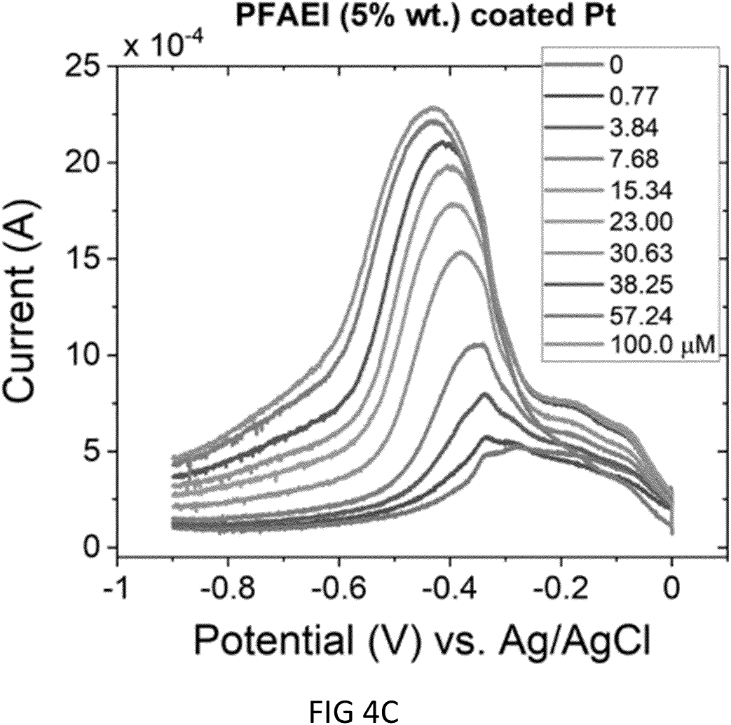

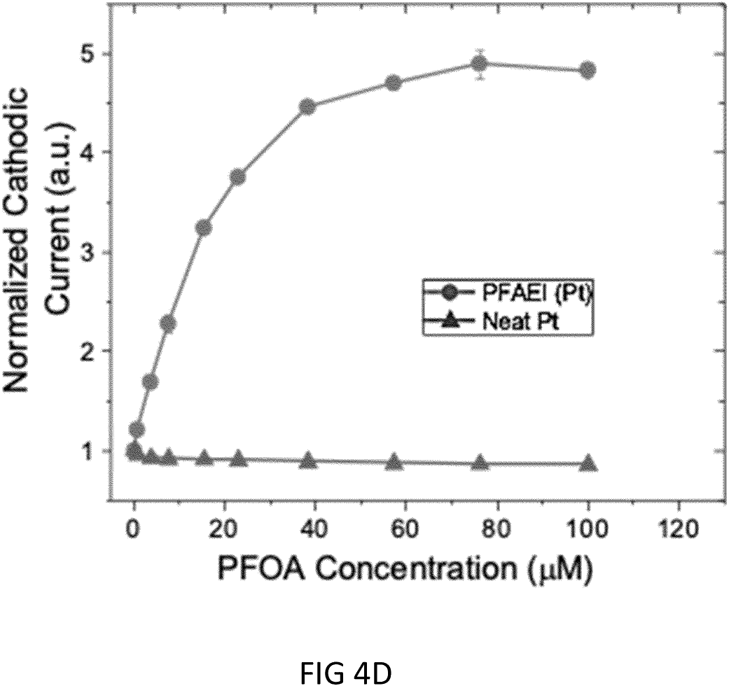

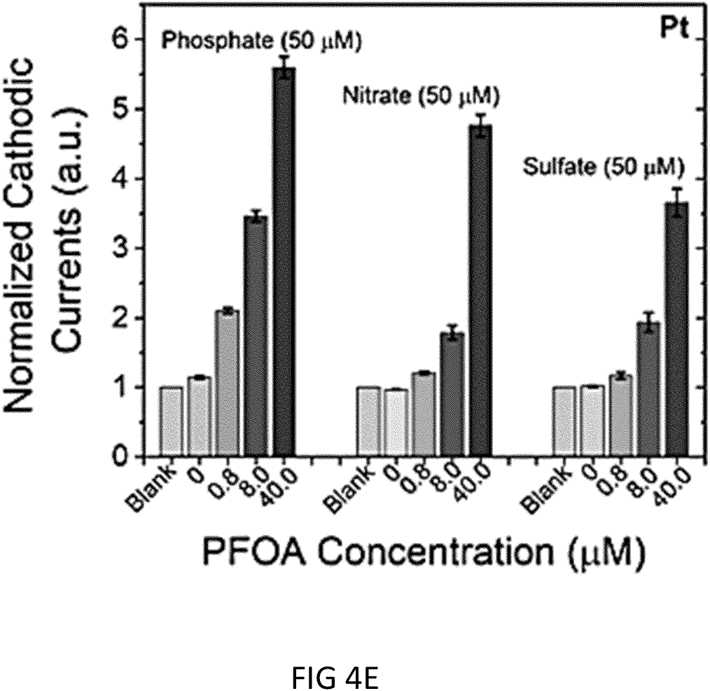

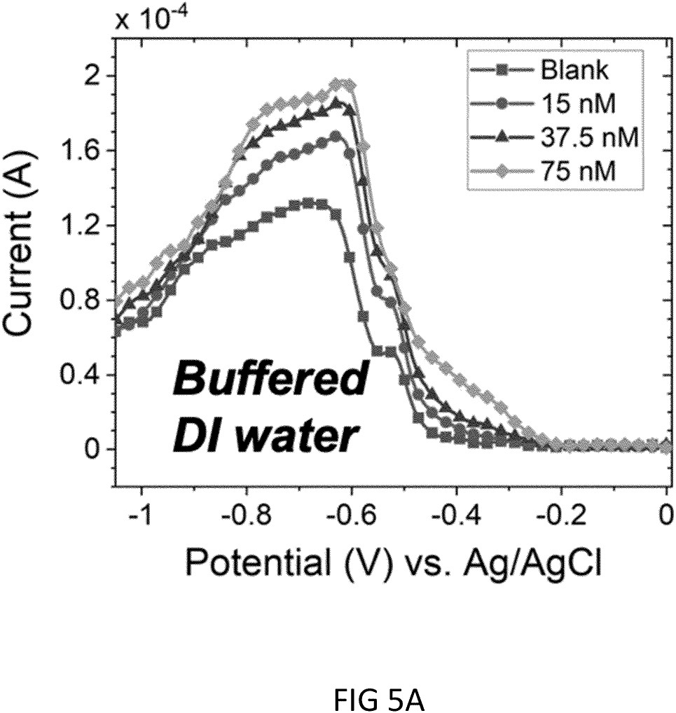

FIG. 4A compares the current response from SWV experiments of a platinum wire working electrode with various ionomer coatings immersed in 7.68 µM PFOA buffered solution. FIG. 4B compares the normalized current at -0.4 V vs Ag/AgCl for the platinum working electrodes with different ionomer coatings. These two plots unequivocally demonstrate that the PFAEI material is the best coating for sensing PFAA. The cation exchange ionomers,Nafion™ and SPEEK, cannot adsorb PFAA anions due to Donnan exclusion. The QPPSf anion exchange ionomer has a hydrocarbon backbone that is not conducive for PFAA adsorption. As discussed earlier, it is the perfluoro-backbone and quaternary ammonium groups of PFAEI that favor PFAA anion adsorption.

After identifying the most effective ionomer coating, SWV was performed with the platinum working electrode coated with PFAEI with solutions that contained different PFOA concentrations (FIG. 4C). The range was 0.77 µM to 100 µM. As the PFOA concentration increased, the current response increased but the peak current slightly shifted from -0.35 V at low concentrations to -0.45 V at high concentrations.

PFAEI coated platinum was selected to be a promising working electrode for PFOA sensing. FIG. 4D shows normalized current response from SWV experiments with a platinum wire working electrode with and without PFAEI coatings as a function of PFOA concentration in solution. The working electrode potential values for the normalized current measurements were -0.40 V vs. Ag/AgCl FIG. 4D shows a commensurate current response with increasing PFOA concentration for the platinum working electrode with a PFAEI coating. Conversely, the working electrode wire with no PFAEI coating experienced a slight decrease in current response when increasing the PFOA concentration in solution. FIG. 4D demonstrates the viability of PFAEI as an effective coating for sensing PFOA with platinum.

In the final experiments with a working electrode wire in a 3-point setup, other interfering anions (e.g., nitrate, phosphate, and sulfate), added as potassium salts, were added to the liquid solution matrix with PFOA and without PFOA to test how these interfering anions affect PFOA sensing. FIG. 4E provides the normalized current response with platinum coated PFAEI working electrodes for 3 liquid samples of varying PFOA concentrations and mixed with 50 µM of interfering anions from potassium salts. These experiments were performed with chronoamperometry, and the current was analyzed at -0.47 V vs Ag/AgCl. In the presence of nitrate, sulfate, and phosphate anions, the normalized current response got larger with increasing PFOA concentrations. Hence, the platinum wire coated PFAEI is a promising candidate for PFOA detection in aqueous solutions with phosphate, sulfate, and nitrate salts. These initial experiments motivated planar, single-substrate sensor studies for PFOA detection in buffered deionized water and drinking water.

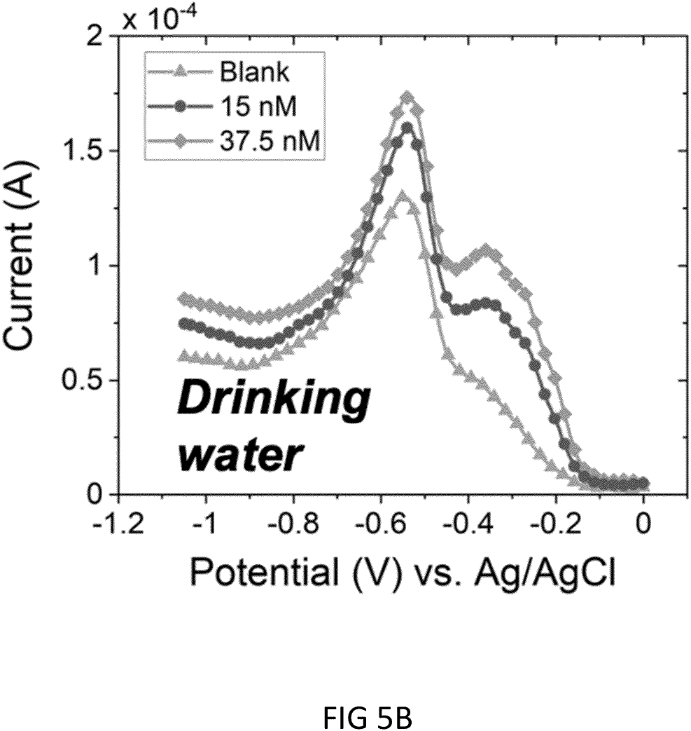

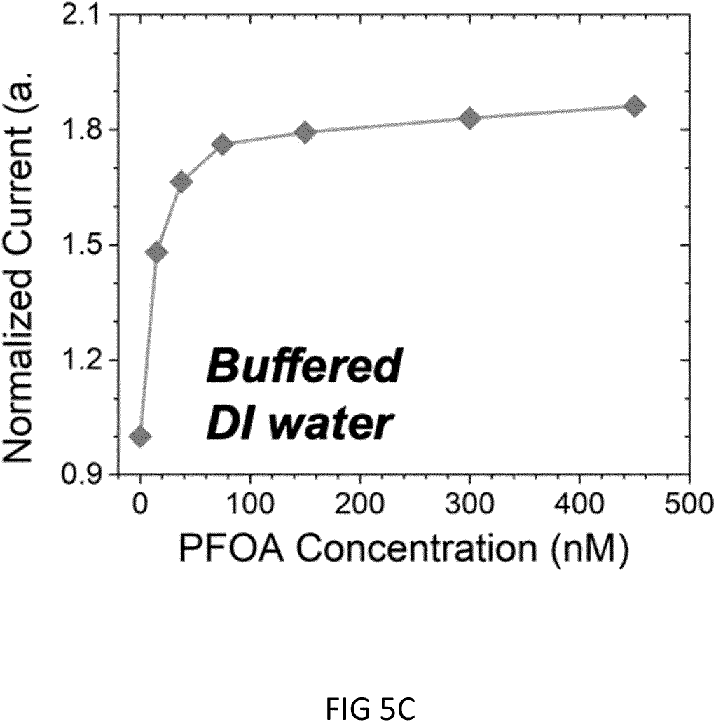

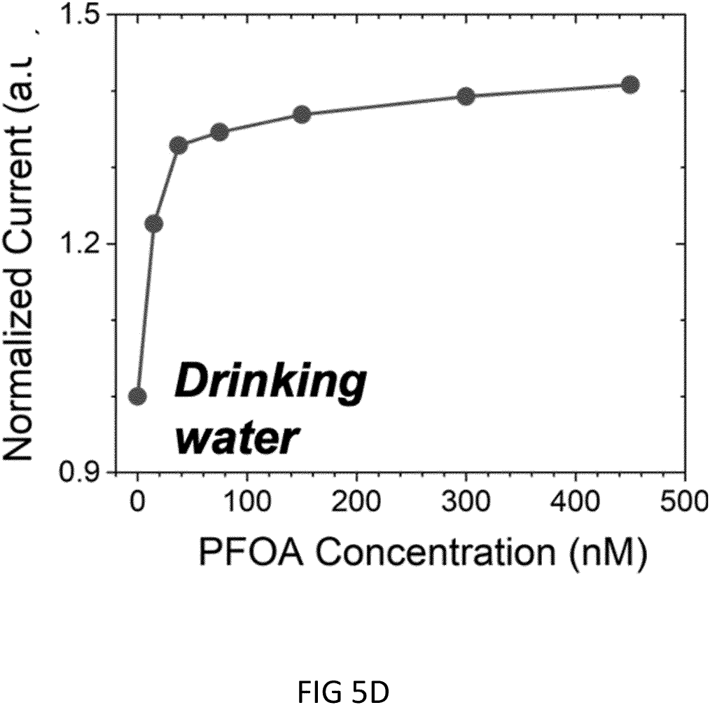

With an appropriate coating and metal electrode identified, SPE electrodes with PFAEI coatings were prepared and assessed for PFOA sensing in DI water with sodium perchlorate/perchloric acid buffer and drinking water using SWV (FIG. 5A and FIG. 5B, respectively). To make the plots clear, SWV traces for a few PFOA concentrations and a solution with no PFOA are only presented. FIGS. 5C and 5D give the normalized current response for the SPE with PFAEI coating with various PFOA concentrations in buffered DI water and drinking water, respectively. Notably, PFOA concentrations were sensed in both buffered DI water and drinking water at concentrations as low as 15 nM PFOA with the Pine SPE coated with PFAEI.

The 15 nM detection limit achieved in this work is about two orders of magnitude higher than EPA health advisory level (0.14 nM) and is roughly 10 times higher than total allowed PFAS concentration limit in drinking water recommended from European Health Commission.³¹ Although the electrochemical sensor reported in this work does not rival the state-of-the-art for LOD in synthetic and actual water solutions, it is a direct method that does not require the addition of redox active probes.

In summary, a judiciously selected ionomer coating, PFAEI, with commercially available SPEs was developed to sense PFOA in model and actual drinking water samples as low as 15 nM.

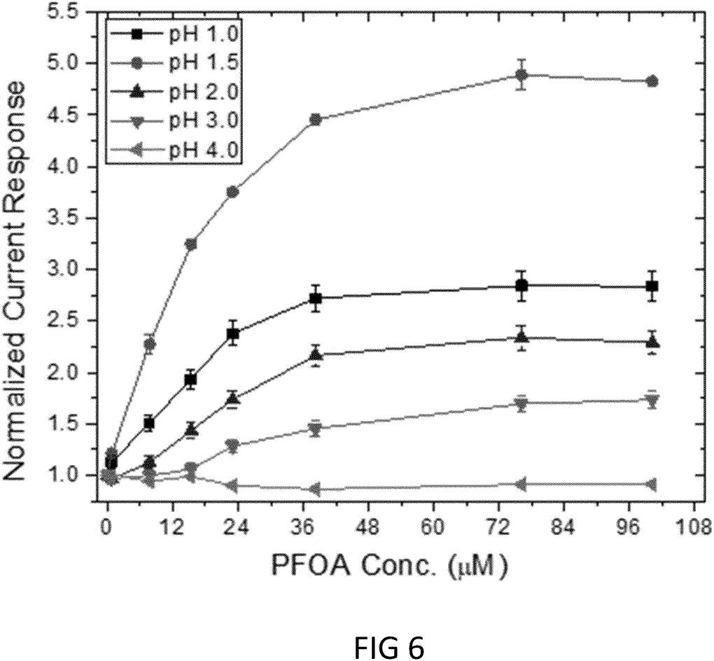

FIGS. 6-18 show additional results obtain in similar experiments using a Pt wire electrode or commercially available DropSens screen printed electrode. More specifically, FIG. 6 shows normalized cathodic current response measured at -0.4 Volt as a function of PFOA concentration using 3 wt% PFAEI coated Pt wire electrodes investigated at different pH values of the supporting electrolyte adjusted using dilute NaOH (0.75 M) or HClO₄.

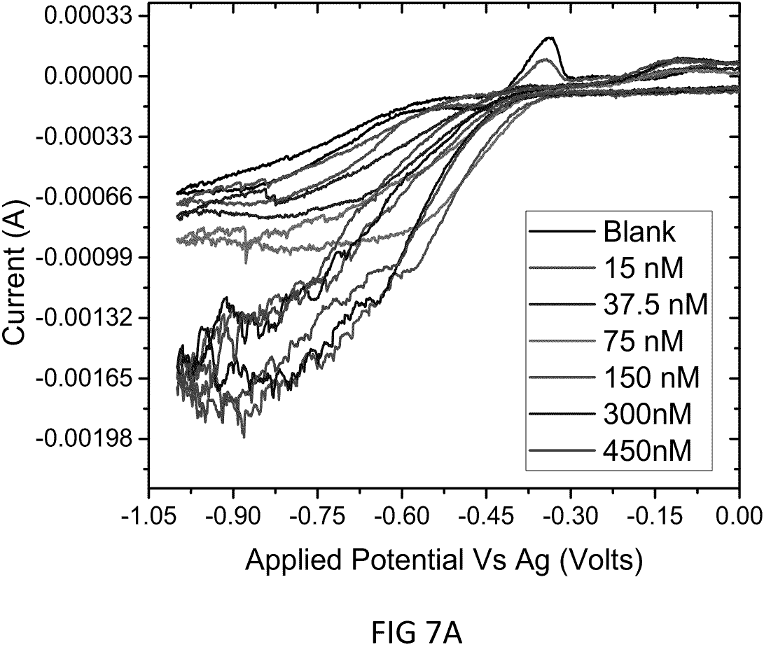

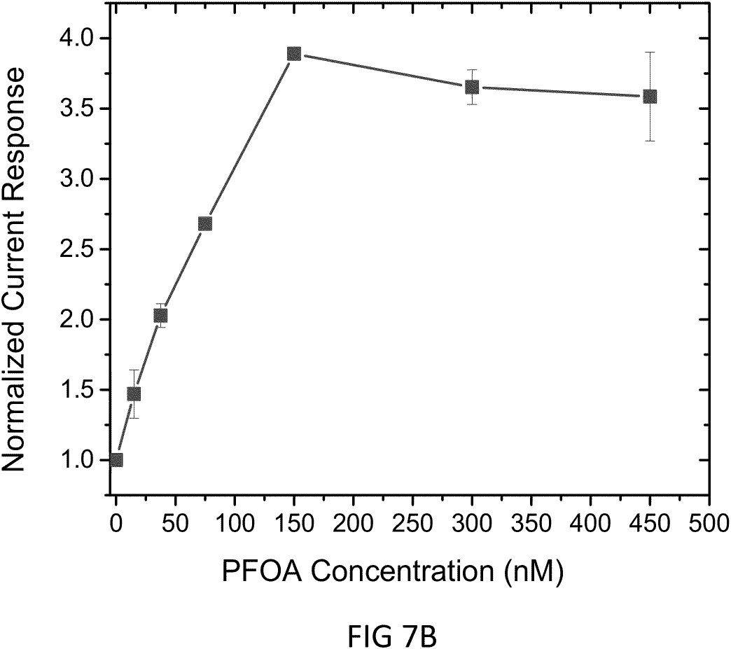

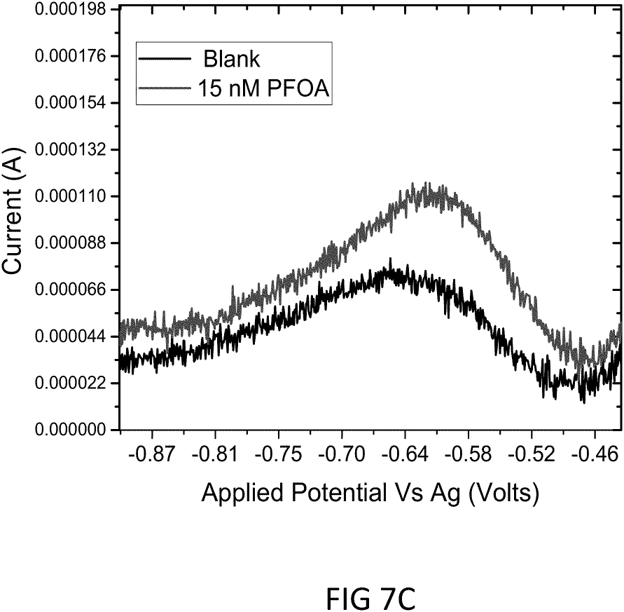

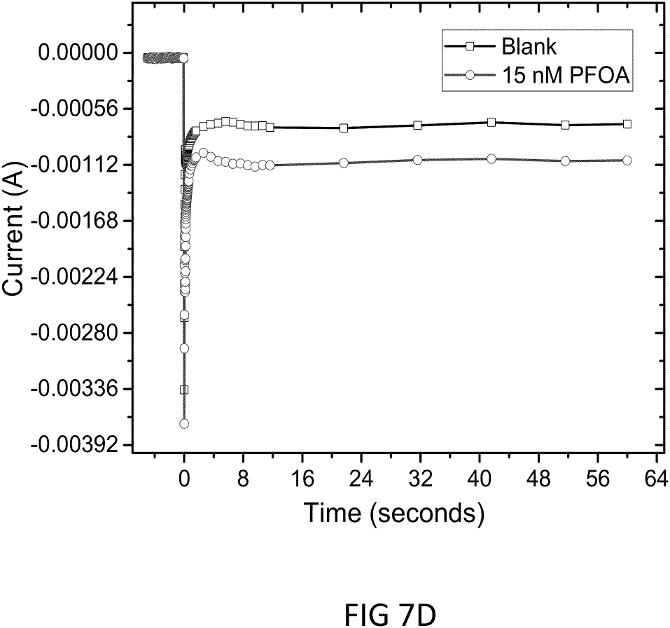

FIGS. 7A and 7B shows cyclic voltammetry (7A), and corresponding normalized current response (7B) measured at -0.68 V Vs Ag as a function of PFOA concentration using PFAEI coated Pt Dropsens electrode (DS-550) in deionized water.

FIGS. 7C and 7D show square wave voltammetry (SWV) results (7C) and chronoamperometry response (7D) seen at applied potential step of -0.68 V Vs Ag in the presence of 15 nM PFOA using PFAEI coated Pt Dropsens electrode (DS-550) in deionized water.

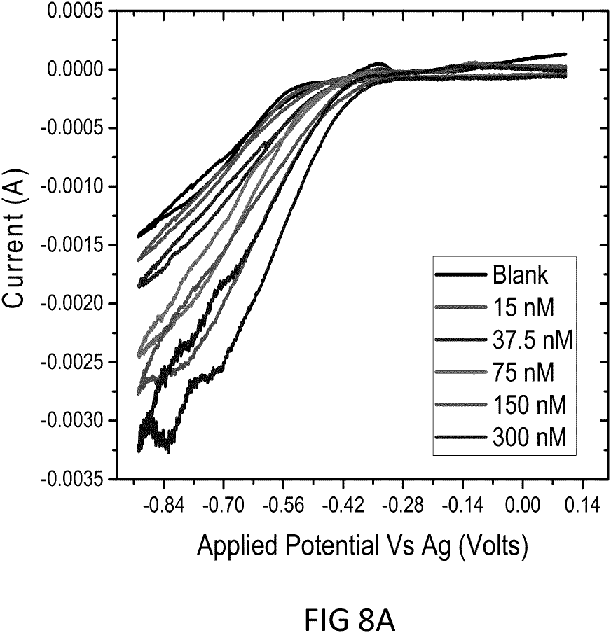

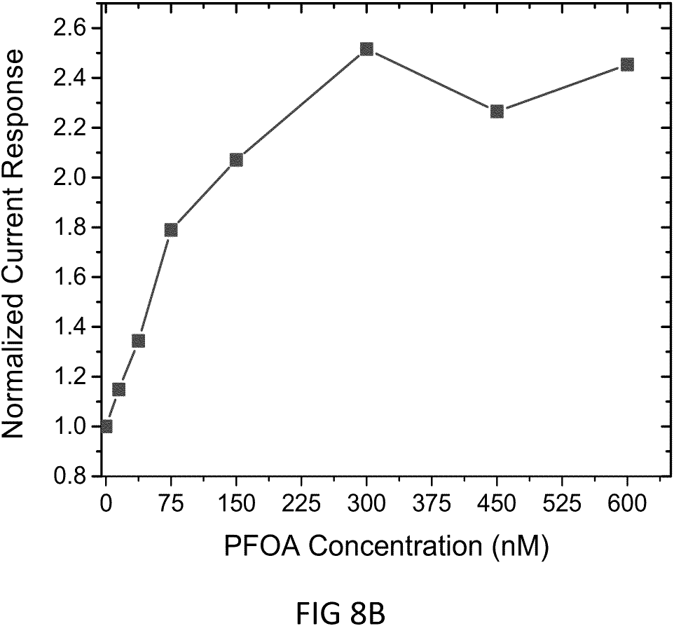

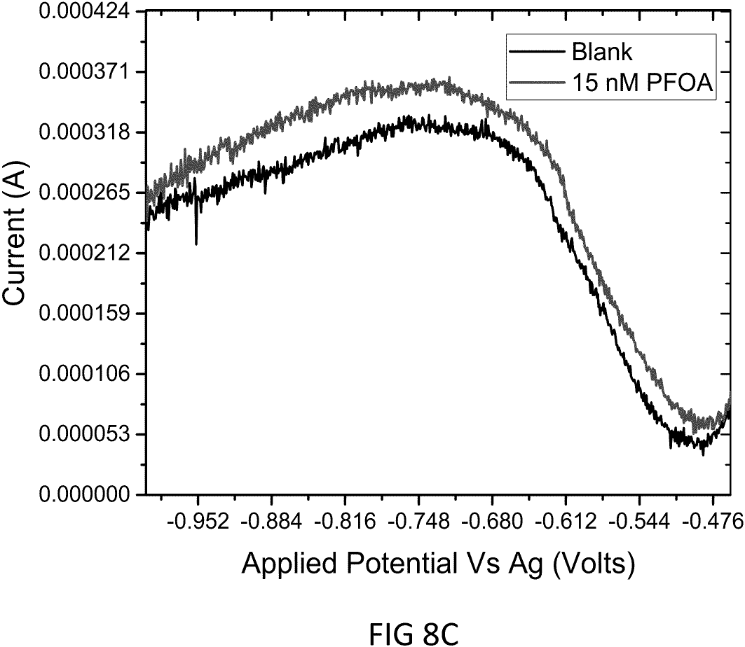

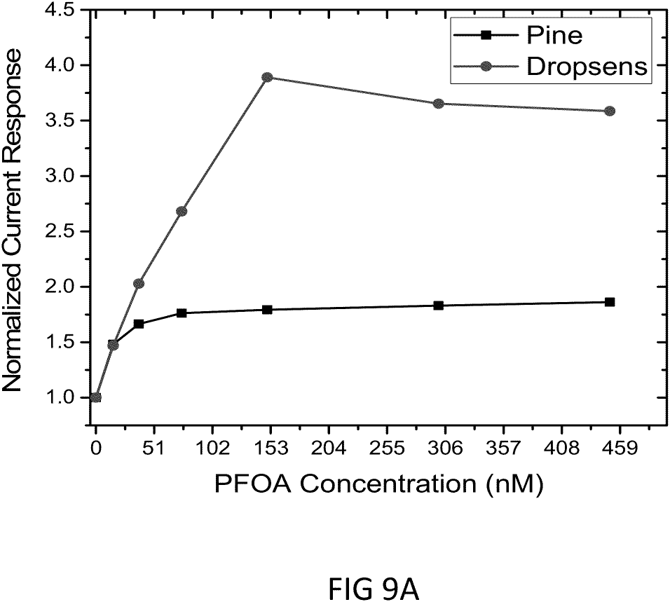

FIGS. 8A and 8B show cyclic voltammetry (8A), and corresponding normalized current response (8B) measured at -0.85 V Vs Ag as a function of PFOA concentration using PFAEI coated Pt Dropsens electrode (DS-550) in drinking water; and

FIG. 8C shows SWV response in simulated drinking water sample spiked with 15 nM PFOA employing PFAEI coated Pt Dropsens (DS-550) electrodes.

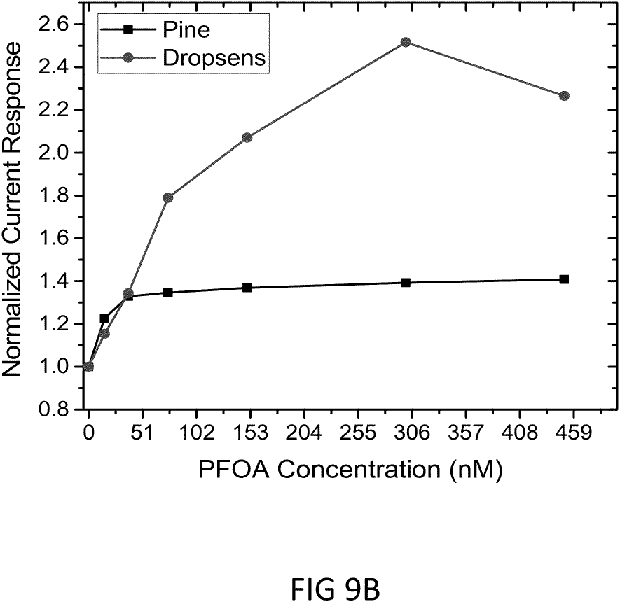

FIGS. 9A and 9B show a comparison of PFOA sensor performance showing limit of detection (LOD) and dynamic range of PFOA detection through normalized cathodic current response obtained from voltammetry measurements as a function of PFOA concentration using PFAEI coated Pt screen-printed electrode configurations from Dropsens (DS-550) and Pine electrodes in deionized water (9A) and in drinking water samples (9B).

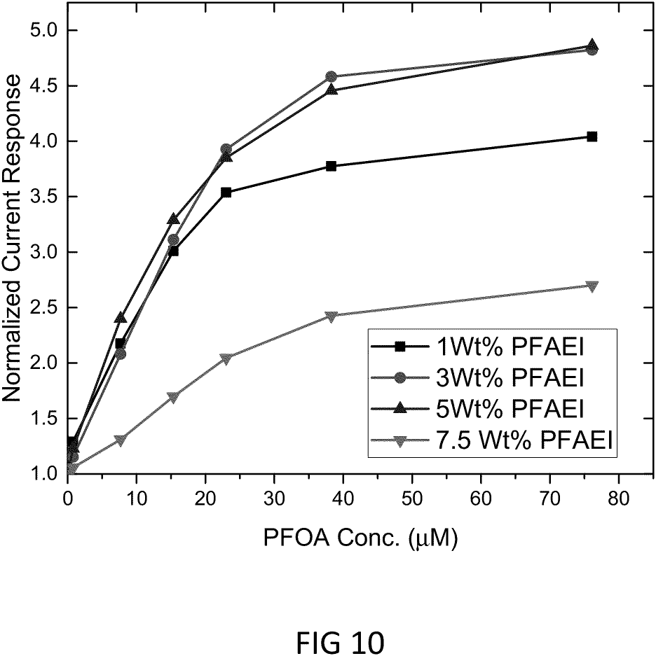

FIG. 10 shows normalized cathodic current responses measured at -0.40 V employing PFAEI coated Pt wire electrodes by using different concentrations of PFAEI ionomer loading solutions in the detection of PFOA at different PFOA concentrations. 3-5 Wt% solution drop casted onto the electrodes provided optimum performance with film thickness measured around 50-70 nm range.

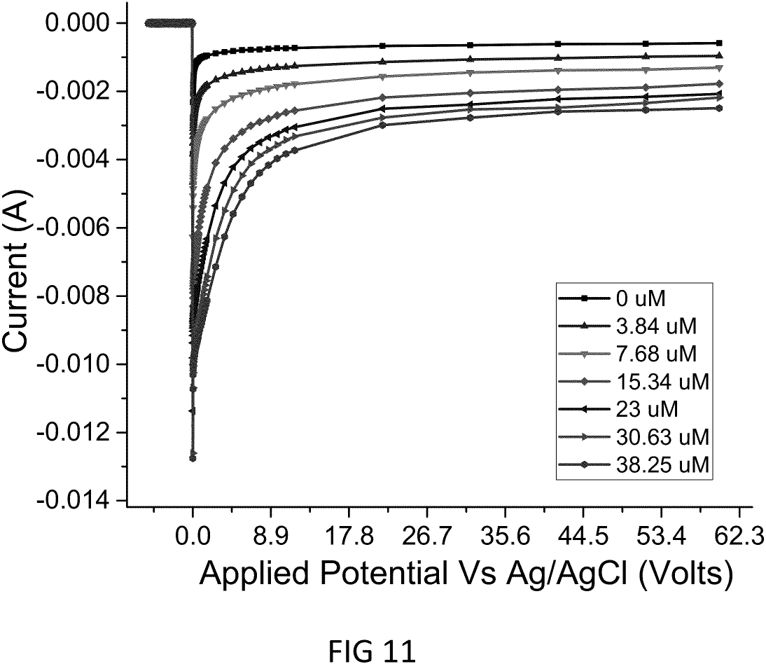

FIG. 11 shows experimentally measured chronoamperometry curves measured at applied potential step of -0.55 V (Left) using PFAEI-Pt wire electrode as PFOA sensor at different PFOA analyte concentrations in deionized water.

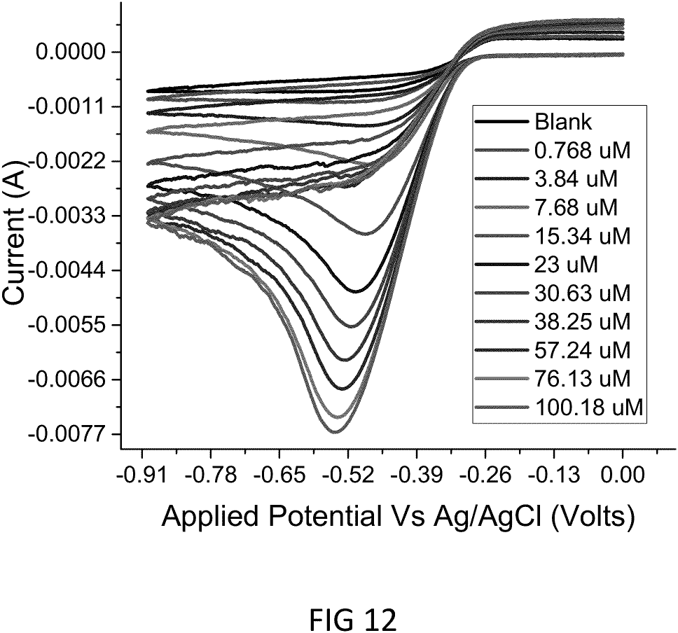

FIG. 12 shows cyclic voltammograms showing increasing current response as function of PFOA concentration in deionized water using PFAEI coated Pt wire electrode sensor.

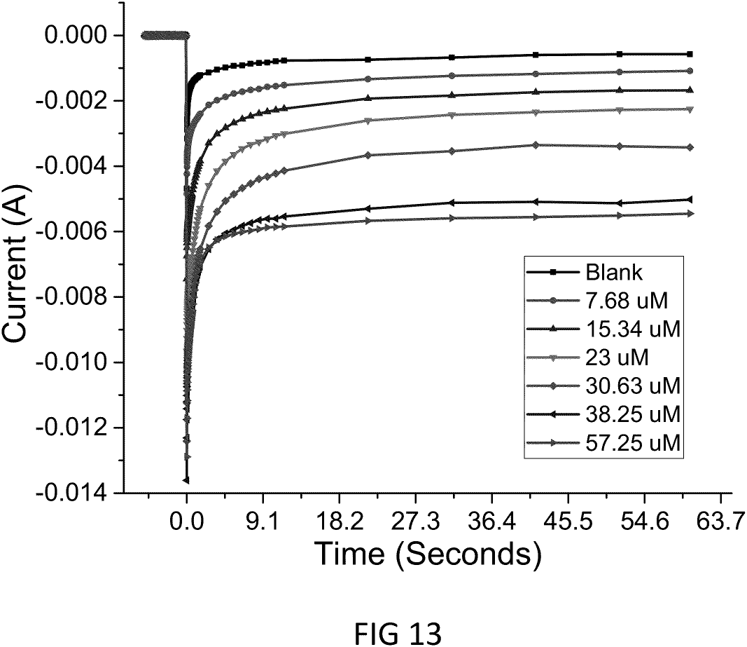

FIG. 13 shows chronoamperometry response measured at applied potential step of -0.8 V Vs Ag/AgCl due to increasing PFOA concentrations using PFAEI coated Pt wire electrodes in drinking water.

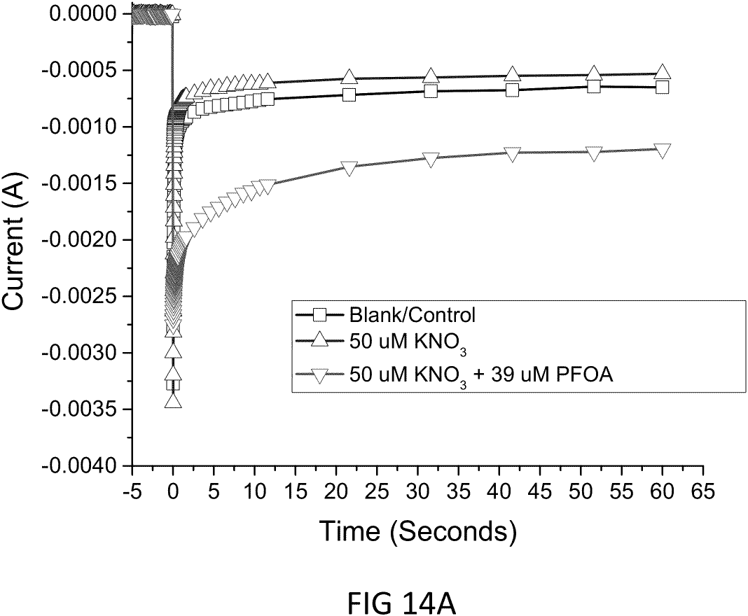

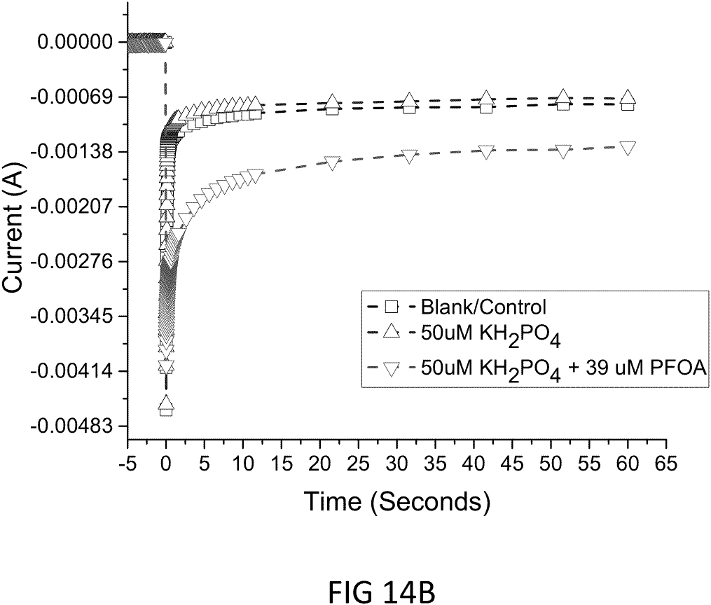

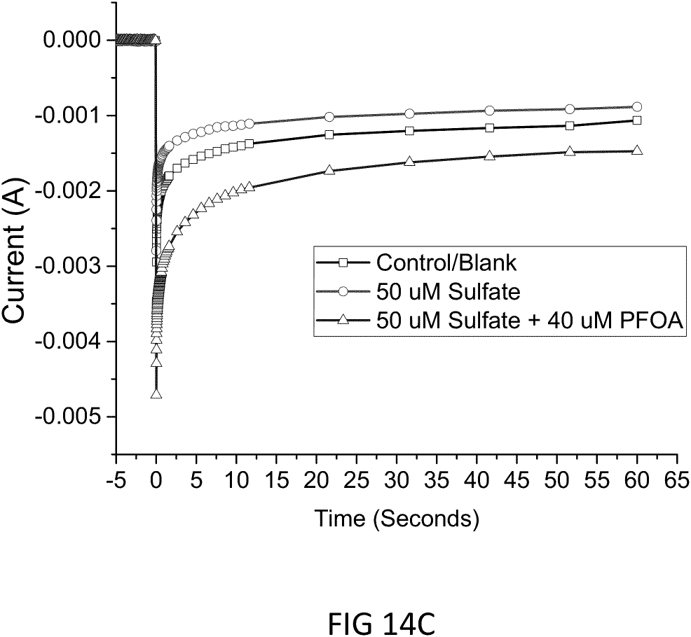

FIGS. 14A-C show chronoamperometry responses measured at applied potential step of -0.85 V Vs Ag/AgCl using PFAEI coated Co wire electrodes in presence of interfering anions with concentrations up to 50 µM of nitrate (NO₃⁻) (14A), orthophosphate (H₂PO₄⁻) (14B), and sulfate (SO₄²⁻) ion (14C) with and without addition of 40 µM PFOA.

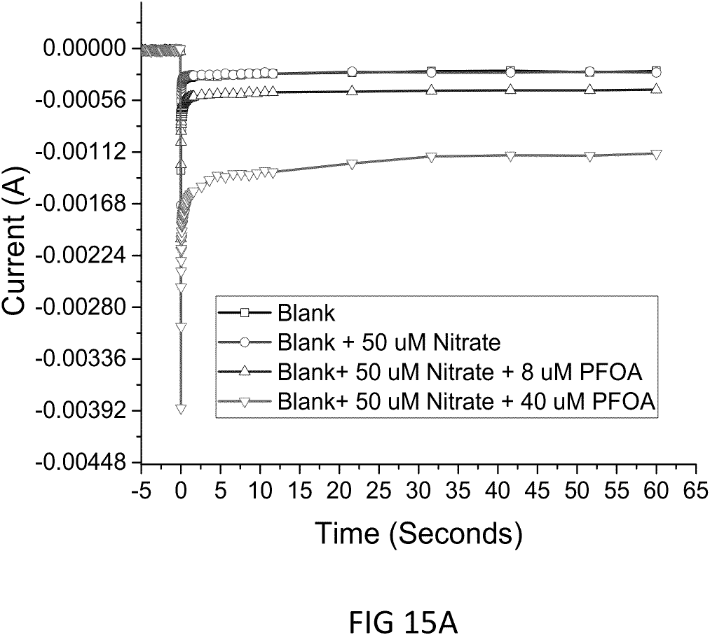

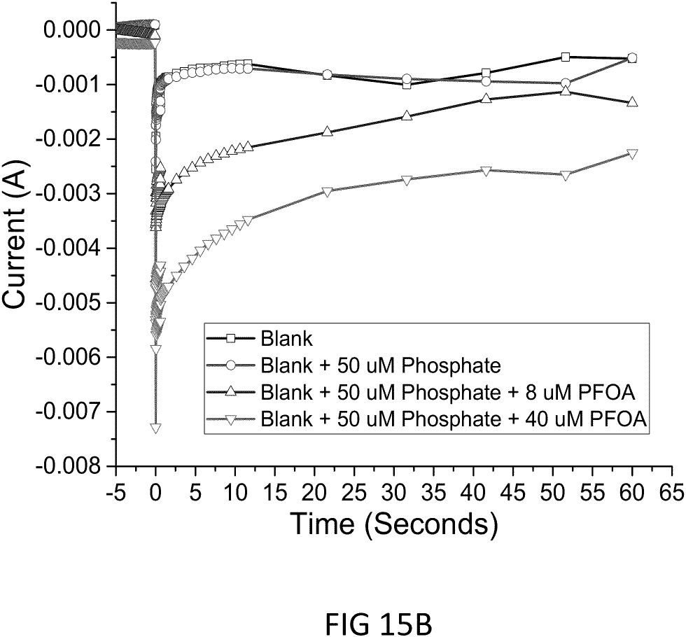

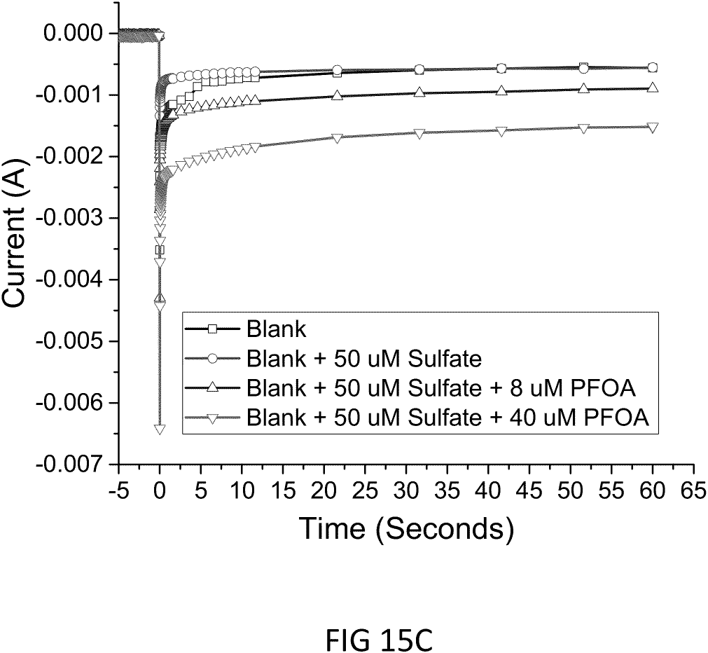

FIGS. 15A-C show Chronoamperometry response measured at applied potential step of -0.47 V Vs Ag/AgCl employing PFAEI coated Pt wire electrodes in presence of 50 µM orthophosphate (H₂PO⁻₄) (15A), nitrate (NO₃) (15B) and sulfate (SO₄²⁻) (15C) as interfering anions with and without addition of 40 µM PFOA.

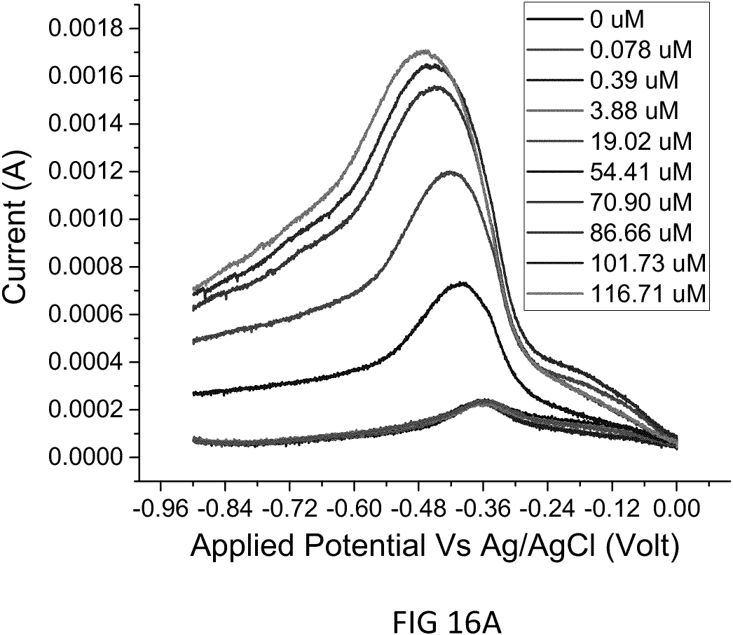

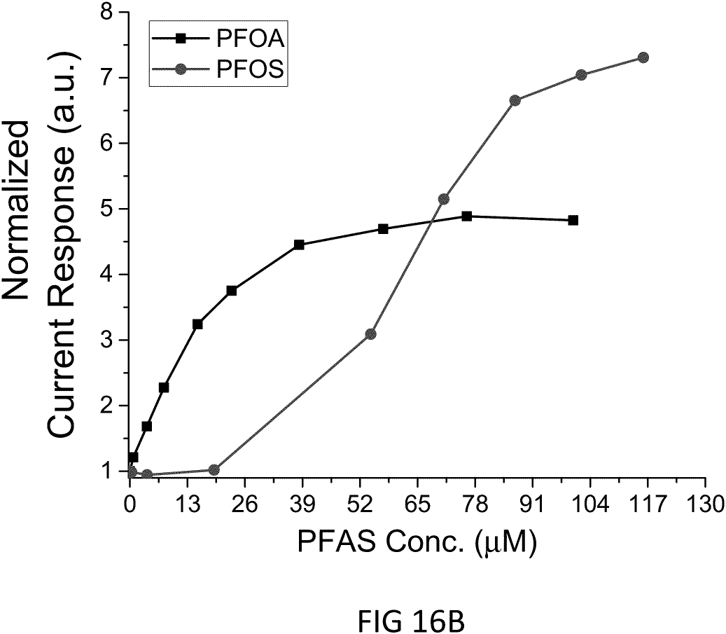

FIGS. 16A and Bshow square wave voltammetry of wire data with PFAEI coating with various PFOS concentrations vs Ag/AgCl (16A) and normalized current responses measured as a function of varying PFOA and PFOS concentrations for a given cathodic peak potential for PFAEI ionomer coated Pt wire electrodes (16B).

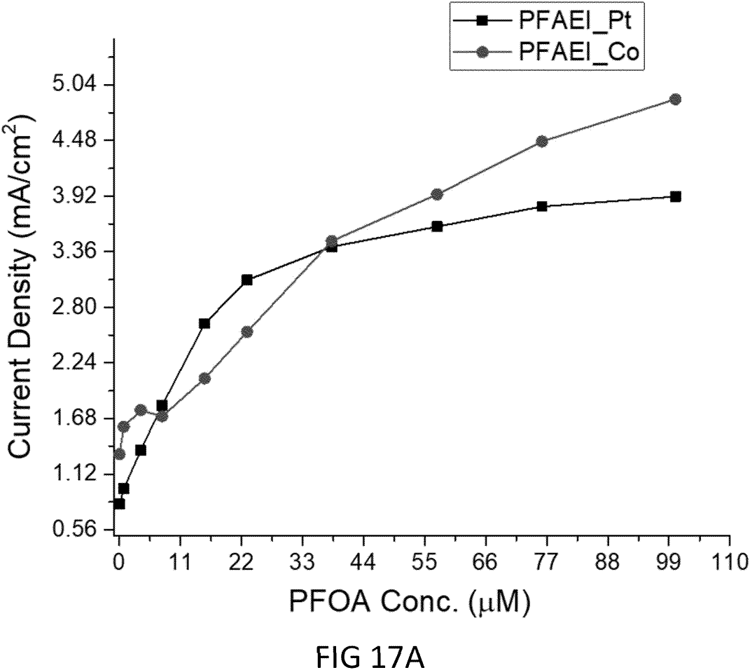

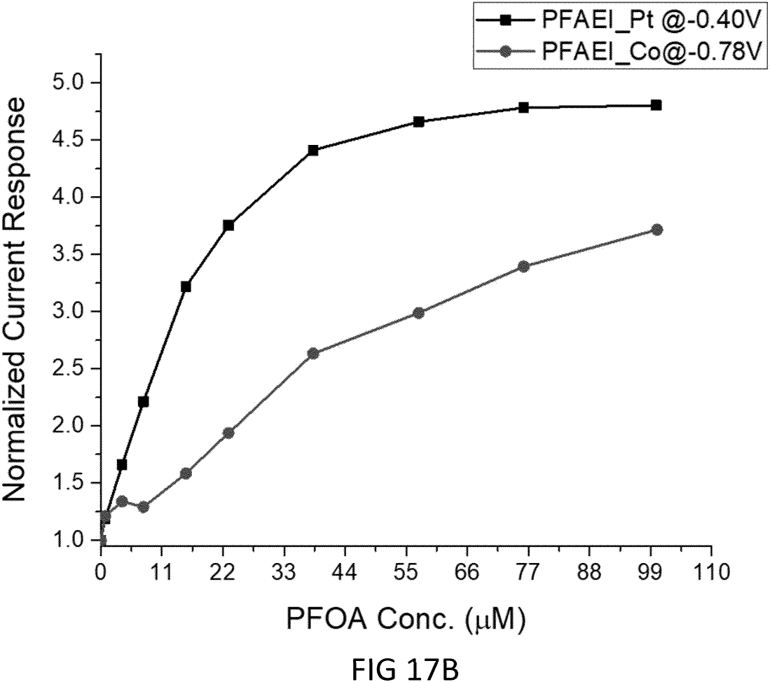

FIGS. 17A and Bshow a comparison of PFAEI coated metal electrode (Co and Pt) sensitivity in PFOA detection using metal wire electrodes. FIG. 17A shows current density after normalization with respect to geometric surface area of wire electrodes. FIG. 17B shows as normalized current response at specified potential observed in PFOA detection as a function of increasing PFOA concentration.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

It is understood that the various embodiments described herein are by way of example only, and are not intended to limit the scope of the invention. For example, many of the materials and structures described herein may be substituted with other materials and structures without deviating from the spirit of the invention. The present invention as claimed may therefore include variations from the particular examples and preferred embodiments described herein, as will be apparent to one of skill in the art. It is understood that various theories as to why the invention works are not intended to be limiting.

What is claimed is:

1. A sensor for measuring perfluoroalkyl acids and/or polyfluoroalkyl acids comprising:
a working electrode; and
a counter electrode;
wherein the working electrode comprises a film disposed on the surface of the working electrode; and
wherein the film comprises a perfluorinated anion exchange ionomer.

2. The sensor for measuring perfluoroalkyl acids and/or polyfluoroalkyl acids of claim 1, wherein the sensor further comprises a reference electrode.

3. The sensor for measuring perfluoroalkyl acids and/or polyfluoroalkyl acids of claim 1, wherein the film has a thickness of 5 to 500 nm.

4. The sensor for measuring perfluoroalkyl acids and/or polyfluoroalkyl acids of claim 1, wherein the perfluorinated anion exchange ionomer comprises a polymer comprising perfluoro groups and at least one cationic group tethered to the polymer comprising the perfluoro groups.

5. The sensor for measuring perfluoroalkyl acids and/or polyfluoroalkyl acids of claim 4, wherein the perfluoro groups are located on a backbone of the polymer, on a side chain or both.

6. The sensor for measuring perfluoroalkyl acids and/or polyfluoroalkyl acids of claim 4, wherein the at least one cationic group is an ammonium, a phosphonium, a sulfonium, an organometallic cation, or a combination thereof.

7. The sensor for measuring perfluoroalkyl acids and/or polyfluoroalkyl acids of claim 4, wherein the cationic group includes one or more of the following structures: 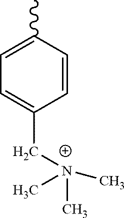 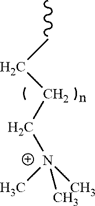 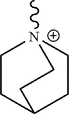 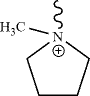 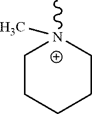 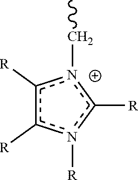 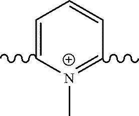 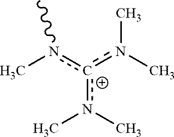 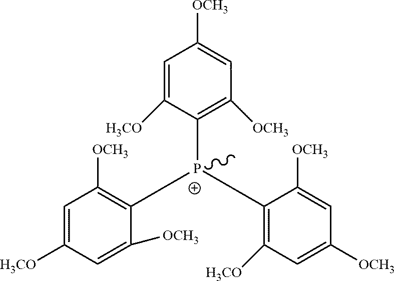 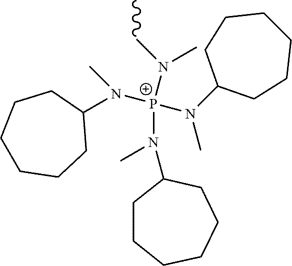 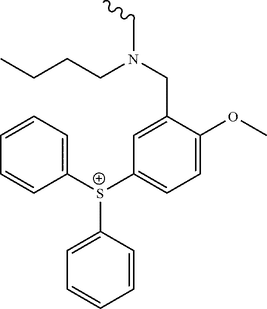 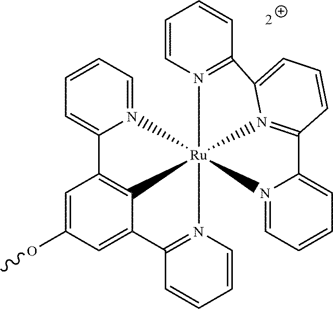 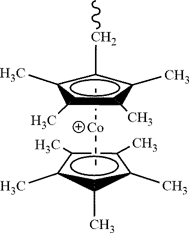
wherein the wavy line indicates the attachment point to the polymer;
each occurrence of R is independently selected from the group consisting of hydrogen, deuterium, fluorine, C₆-C₁₈ aryl, and C₁-C₁₀ alkyl; and
n is an integer having a value between 1 and 100.

8. The sensor for measuring perfluoroalkyl acids and/or polyfluoroalkyl acids of claim 1, wherein the working electrode comprises cobalt, platinum, palladium, gold, iron, aluminum, copper, nickel, zinc, silver, or an alloy comprising one or more of the foregoing.

9. The sensor for measuring perfluoroalkyl acids and/or polyfluoroalkyl acids of claim 1, wherein the working electrode, counter electrode and optional reference electrode are screen printed on a ceramic, glass, quartz, polymer, or hydrogel substrate.

10. The sensor for measuring perfluoroalkyl acids and/or polyfluoroalkyl acids of claim 1, wherein the working electrode further comprises a nanoscale electrocatalyst.

11. The sensor for measuring perfluoroalkyl acids and/or polyfluoroalkyl acids of claim 1, wherein the working electrode and counter electrode are interdigitated.

12. The sensor for measuring perfluoroalkyl acids and/or polyfluoroalkyl acids of claim 1, wherein the working electrode comprises nanoparticles.

13. A containment vessel comprising the sensor for measuring perfluoroalkyl acids and/or polyfluoroalkyl acids of claim 1.

14. The containment vessel of claim 13, wherein the vessel comprises a series of point sensors functionally connected to an alarm system.

15. A method of detecting perfluoroalkyl acids and/or polyfluoroalkyl acids comprising the steps of:
providing a sample for measurement;
contacting the sample with the sensor of claim 1; and
determining the presence of perfluoroalkyl acids based on the response of the sensor to the sample.

16. A method of detecting perfluoroalkyl acids and/or polyfluoroalkyl acids comprising the steps of:
providing a sample for measurement;
contacting the sample with a sensor; and determining the presence of perfluoroalkyl acids based on the response of the sensor to the sample.

17. The method of claim 16, further comprising the step of acidifying the sample to a pH of 1 to 3.

18. The method of claim 16, wherein the sensor for measuring perfluoroalkyl acids and/or polyfluoroalkyl acids comprises:
a working electrode; and
a counter electrode;
wherein the working electrode comprises a film disposed on the surface of the working electrode; and
wherein the film comprises a perfluorinated anion exchange ionomer.

19. The method of claim 16, wherein the sensor further comprises a reference electrode.

20. The method of claim 16, wherein the perfluorinated anion exchange ionomer comprises a polymer comprising perfluoro groups and at least one cationic group tethered to the polymer comprising the perfluoro groups.
